(19) United States
(12) Patent Application Publication
(10) Pub. No.: US 2017/0094450 A1
(43) Pub. Date: Mar. 30, 2017

(54) CROWDSOURCING ACTIVITY DETECTION FOR GROUP ACTIVITIES

(71) Applicant: Apple Inc., Cupertino, CA (US)

(72) Inventors: Xiaoyuan TU, Cupertino, CA (US); Anil K. KANDANGATH, Cupertino, CA (US)

(73) Assignee: Apple Inc., Cupertino, CA (US)

(21) Appl. No.: 15/273,054

(22) Filed: Sep. 22, 2016

Related U.S. Application Data
(60) Provisional application No. 62/235,161, filed on Sep. 30, 2015.

(51) Int. Cl. H04W 4/00; G08C 17/02; G01S 19/13; A61B 5/024; A61B 5/11; A61B 5/00; H04W 4/02; H04L 29/08

(52) U.S. Cl. CPC H04W 4/008; H04W 4/027; H04W 4/02; G08C 17/02; H04L 67/22; H04W 4/026; A61B 5/02438; A61B 5/1118; A61B 5/7275; A61B 5/1112; G01S 19/13; H04W 84/18

(57) ABSTRACT

In one aspect, the present disclosure relates to a method, including determining, by a wearable device, predicting, by a first wearable device, a first predicted activity of the first user using motion data received by motion sensors of the first wearable device; estimating, a first confidence level of the first predicted activity; receiving, by the first wearable device over a wireless communication channel from a second wearable device, a second predicted activity of a second user and a second confidence level of the second predicted activity; comparing the first predicted activity and the first confidence level with the second predicted activity and the second confidence level; and determining a first activity classification for the first user to be the second predicted activity when a second average confidence level associated with the second predicted activity is greater than a first average confidence level associated with the first predicted activity.

### CROSS REFERENCE TO RELATED APPLICATIONS

This application claims priority to U.S. Provisional Pat. App. No. 62/235,161 titled “Crowdsourcing Activity Detection For Group Activities”, filed Sep. 30, 2015, which is incorporated by reference herein in its entirety.

### FIELD

The present disclosure relates generally to activity detection during group activities and, more particularly, to techniques for crowdsourcing activity detection using motion data or other information from wearable devices within a group.

### BACKGROUND

A wearable device can contain motion sensors to collect data about the wearable device's position and orientation in space and to track changes to the wearable device's position and orientation over time. Because a user can wear the wearable device, the motion data can provide information about the user's movement or activity. By analyzing the motion data, the wearable device may be capable of inferring—with varying degrees of accuracy—the type of movement or activity in which the user is engaged.

For example, when a user is running, the user's arms are typically swinging back and forth over a particular distance and at a particular frequency. If the user wears the wearable device on the user's wrist, the wearable device may be able to make range and frequency measurements and infer that the user is running.

Depending on the analysis technique and the motion data, the wearable device may also determine a confidence level for its prediction. For example, the wearable device could determine that there is an approximately 80% likelihood that the user is running as opposed to an approximately 20% likelihood that the user is walking.

Making accurate predictions has several benefits. For example, the wearable device may be able to estimate calorie expenditure more accurately if it knows the type of activity. However, the activity predictions could vary in degrees of accuracy from one moment to the next, or from one activity session to the next. Under some conditions, the wearable device may be unable to make a prediction, or it may make an incorrect prediction by identifying an activity different from the user's actual activity.

Sometimes, the user may be participating in a group activity. For example, the user could be running with a running club, and other members on the run could be wearing their own wearable devices. Even though each member of the group is running together, each member's device makes its own individualized prediction to detect and classify its user's activity.

For example, a first user's wearable device may correctly predict that the user is running with a high confidence level, a second user's wearable device may correctly predict that the second user is running with a low confidence level, and a third user's wearable device may incorrectly predict that the third user is walking with a low confidence level. Thus, the wearable devices for each of the three users who are running together could provide their users with varying information and varying quality of service.

### SUMMARY

Embodiments of the present disclosure include a wearable device and techniques for accurately detecting a user's activity by crowdsourcing motion data, activity predictions, or other information from wearable devices of other users participating in a group activity. In some embodiments, the wearable device may be worn on a wrist, such as a watch or bracelet, and it may include one or more microprocessors, a display, and a variety of sensors, such as a heart rate sensor and one or more motion sensors.

Embodiments of the present disclosure may provide accurate, individualized activity tracking throughout a person's day, and across a variety of activities. Some embodiments may calibrate a wearable device for an individual without necessarily relying on self-reporting about physical activity.

In some embodiments, the motion sensors may include, for example, an accelerometer, a gyroscope, a barometer or altimeter, a magnetometer or compass, etc. The wearable device may also include a motion coprocessor, which may be optimized for low-power, continuous motion sensing and processing.

In some embodiments, the wearable device may be capable of communicating with a companion device. The wearable device may communicate with a companion device wirelessly, e.g., via a Wi-Fi, cellular, or Bluetooth connection, or similar wireless or wired communication method. The companion device may be a mobile device, such as a smartphone, tablet, etc, which may include additional sensors. The additional sensors in the companion device may include a Global Positioning System (GPS) sensor, accelerometer, gyroscope, barometer or altimeter, motion coprocessor, or any other sensor or combination of sensors. The companion device may, for example, communicate location information based on data from the GPS sensor to the wearable device.

In some embodiments, a first wearable device may be capable of communicating with other wearable devices. The first wearable device may communicate with other wearable devices wirelessly, e.g., via a WiFi, cellular, or Bluetooth connection, or similar wireless or wired communication method. In some embodiments, some of the other wearable devices may include different hardware or firmware and may communicate using a common inter-device protocol and implement a given application programming interface (API). The first wearable device may, for example, communicate motion data, activity predictions, or other information to the other wearable devices. The first wearable device may also be configured to receive information in kind from the other wearable devices.

In one aspect, the present disclosure relates to a method, including determining, by a wearable device, predicting, by a first wearable device, a first predicted activity of the first user using motion data received by motion sensors of the first wearable device; estimating, by the first wearable device, a first confidence level of the first predicted activity; receiving, by the first wearable device over a wireless communication channel from a second wearable device, a second predicted activity of a second user and a second confidence level of the second predicted activity; comparing, by the first wearable device, the first predicted activity and the first confidence level with the second predicted activity and the second confidence level; and determining, by the first wearable device, a first activity classification for the first user to be the second predicted activity when a second average confidence level associated with the second predicted activity is greater than a first average confidence level associated with the first predicted activity.

Other features and advantages will become apparent from the following detailed description and drawings.

### BRIEF DESCRIPTION OF THE DRAWINGS

In order to facilitate a fuller understanding of the present disclosure, reference is now made to the accompanying drawings, in which like elements are referenced with like numerals. These drawings should not be construed as limiting the present disclosure, but are intended to be illustrative only.

FIG. 1 shows a wearable device in accordance with an embodiment of the present disclosure.

FIG. 2 depicts a block diagram of a wearable device in accordance with an embodiment of the present disclosure.

FIG. 3 shows a companion device in accordance with an embodiment of the present disclosure.

FIG. 4 shows a schematic representation of a group activity in accordance with an embodiment of the present disclosure.

FIG. 5 shows a schematic representation of a group activity in accordance with an embodiment of the present disclosure.

FIG. 6 shows a schematic representation of a group activity in accordance with an embodiment of the present disclosure.

FIG. 7 shows a schematic representation of a group activity in accordance with an embodiment of the present disclosure.

FIG. 8 shows a schematic representation of a group activity in accordance with an embodiment of the present disclosure.

FIG. 9 shows a schematic representation of a group activity in accordance with an embodiment of the present disclosure.

FIG. 10 depicts an activity classification method in accordance with an embodiment of the present disclosure.

FIG. 11 depicts an activity classification method in accordance with an embodiment of the present disclosure.

### DESCRIPTION

There is growing interest to assess and monitor one's health or fitness and physical activity. The present disclosure describes a wearable device that may be configured to crowdsource activity detection using motion data, activity predictions, or other information from wearable devices of other members of a group in a group activity.

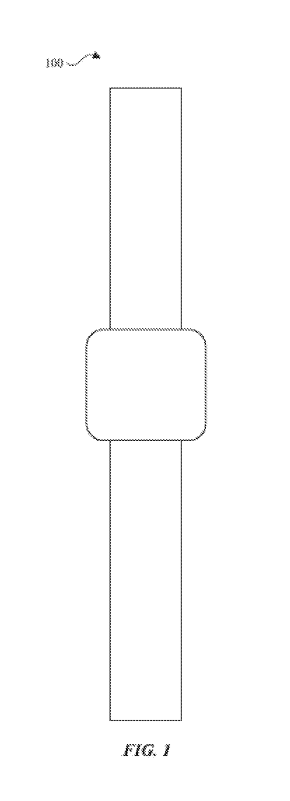

FIG. 1 shows an example of a wearable device 100 in accordance with an embodiment of the present disclosure. In some embodiments, the wearable device 100 may be a wearable device, such as a watch configured to be worn around an individual's wrist, glasses, clothing, or other accessories with integrated electronics. The wearable device 100 may be calibrated according to physical attributes of the individual and physical activity by the individual user who is wearing the wearable device 100. For example, the wearable device 100 may track physical attributes of the user (e.g., age, sex, weight, body mass index, etc.), physical activity or fitness levels (e.g., aerobic capacity, resting heart rate, etc.), physical activity participation statistics (e.g., times of day, durations, types of activities, etc.), and other information about the user.

FIG. 2 depicts a block diagram of example components that may be found within the wearable device 100 in accordance with an embodiment of the present disclosure. These components may include a heart rate sensing module 210, a motion sensing module 220, a display module 230, and an interface module 240. While modules 210, 220, 230, and 240 are described as separate modules, these modules may be combined into fewer modules or separated into more modules. The wearable device 100 may include any other suitable modules or combination of modules.

The heart rate sensing module 210 may include or may be in communication with a heart rate sensor. The wearable device 100 can measure an individual's current heart rate from the heart rate sensor. The heart rate sensor may also be configured to determine a confidence level indicating a relative likelihood of an accuracy of a given heart rate measurement. In other embodiments, a traditional heart rate monitor may be used and may communicate with the wearable device 100 through a near field communication method (e.g., Bluetooth).

The wearable device 100 may also include the motion sensing module 220. The motion sensing module 220 may include one or more motion sensors, such as an accelerometer or a gyroscope. In some embodiments, the accelerometer may be a three-axis, microelectromechanical system (MEMS) accelerometer, and the gyroscope may be a three-axis MEMS gyroscope. A microprocessor (not shown) or motion coprocessor (not shown) of the wearable device 100 may receive motion information from the motion sensors of the motion sensing module 220 to track acceleration, rotation, position, or orientation information of the wearable device 100 in six degrees of freedom through three-dimensional space.

In some embodiments, the motion sensing module 220 may include other types of sensors in addition to accelerometers and gyroscopes. For example, the motion sensing module 220 may include an altimeter or barometer, or other types of location sensors, such as a GPS sensor.

The wearable device 100 may also include a display module 230. Display module 230 may be a screen, such as a crystalline (e.g., sapphire) or glass screen, configured to provide output to the user. In some embodiments, the display module 230 may include a touchscreen to receive input form the user via touch, and a haptic feedback mechanism (e.g., electromagnets, motors, pressure sensors, etc.) to provide feedback via the user's sense of touch. For example, display 230 may be configured to display a current heart rate or a daily average energy expenditure. Display module 230 may receive input from the user to select, for example, which information should be displayed, or whether the user is beginning a physical activity (e.g., starting a session) or ending a physical activity (e.g., ending a session), such as a running session or a cycling session. In some embodiments, the wearable device 100 may present output to the user in other ways, such as by producing sound with a speaker (not shown), and the wearable device 100 may receive input from the user in other ways, such as by receiving voice commands via a microphone (not shown).

In some embodiments, the wearable device 100 may communicate with external devices via interface module 240, including a configuration to present output to a user or receive input from a user. Interface module 240 may be a wireless interface. The wireless interface may be a standard Bluetooth (IEEE 802.15) interface, such as Bluetooth v4.0, also known as “Bluetooth low energy.” In other embodiments, the interface may operate according to a cellphone network protocol such as the LTE, CDMA, GSM, etc. cellular protocols or a Wi-Fi (IEEE 802.11) protocol. In other embodiments, interface module 240 may include wired interfaces, such as a headphone jack or bus connector (e.g., Lightning, Thunderbolt, USB, etc.).

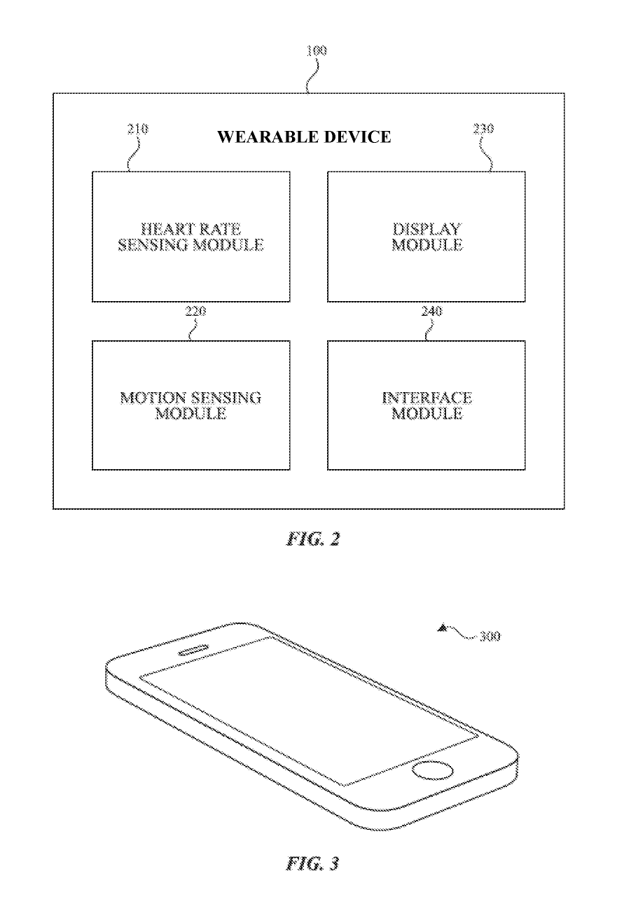

The wearable device 100 may be configured to communicate with a companion device 300 (FIG. 3), such as a smartphone, as described in more detail herein. In some embodiments, the wearable device 100 may be configured to communicate with other external devices, such as a notebook or desktop computer, tablet, headphones, Bluetooth headset, etc.

The modules described above are examples, and embodiments of the wearable device 100 may include other modules not shown. For example, the wearable device 100 may include one or more microprocessors (not shown) for processing heart rate data, motion data, other information in the wearable device 100, or executing instructions for firmware or apps stored in a non-transitory processor-readable medium such as a memory module (not shown). Additionally, some embodiments of the wearable device 100 may include a rechargeable battery (e.g., a lithium-ion battery), a microphone or a microphone array, one or more cameras, one or more speakers, a watchband, a crystalline (e.g., sapphire) or glass-covered scratch-resistant display, water-resistant casing or coating, etc.

FIG. 3 shows an example of a companion device 300 in accordance with an embodiment of the present disclosure. The wearable device 100 may be configured to communicate with the companion device 300 via a wired or wireless communication channel (e.g., Bluetooth, Wi-Fi, etc.). In some embodiments, the companion device 300 may be a smartphone, tablet, or similar portable computing device. The companion device 300 may be carried by the user, stored in the user's pocket, strapped to the user's arm with an armband or similar device, placed in a mounting device, or otherwise positioned within communicable range of the wearable device 100.

The companion device 300 may include a variety of sensors, such as location and motion sensors (not shown). When the companion device 300 may be optionally available for communication with the wearable device 100, the wearable device 100 may receive additional data from the companion device 300 to improve or supplement its calibration or calorimetry processes. For example, in some embodiments, the wearable device 100 may not include a GPS sensor as opposed to an alternative embodiment in which the wearable device 100 may include a GPS sensor. In the case where the wearable device 100 may not include a GPS sensor, a GPS sensor of the companion device 300 may collect GPS location information, and the wearable device 100 may receive the GPS location information via interface module 240 (FIG. 2) from the companion device 300.

In another example, the wearable device 100 may not include an altimeter or barometer, as opposed to an alternative embodiment in which the wearable device 100 may include an altimeter or barometer. In the case where the wearable device 100 may not include an altimeter or barometer, an altimeter or barometer of the companion device 300 may collect altitude or relative altitude information, and the wearable device 100 may receive the altitude or relative altitude information via interface module 240 (FIG. 2) from the companion device 300.

As explained above, a wearable device may use motion data to predict a user's activity. Examples of activities may include, but are not limited to, walking, running, cycling, swimming, etc. The wearable device may also be able to predict or otherwise detect when a user is sedentary (e.g., sleeping, sitting, standing still, driving, etc.). The wearable device may use a variety of motion data, including, in some embodiments, motion data from a companion device.

For example, the wearable device may detect that the user's arm is swinging back and forth with a range of motion, angle of motion, and frequency typically exhibited when the user is running. The wearable device may also use GPS data (e.g., from the GPS sensor of a companion device) to estimate that the user's speed is typical for when the user is running. Thus, the wearable device may predict or otherwise determine that the user is running.

The wearable device may predict the user's activity. The wearable device may also estimate a confidence level (e.g., percentage likelihood, degree of accuracy, etc.) associated with a particular prediction (e.g., 90% likelihood that the user is running) or predictions (e.g., 60% likelihood that the user is running and 40% likelihood that the user is walking).

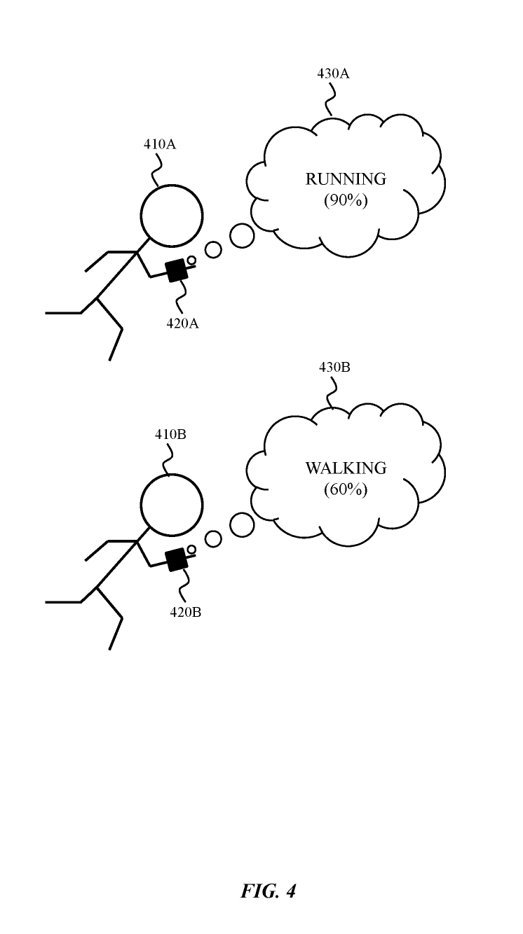

FIG. 4 shows a schematic representation of a group activity in accordance with an embodiment of the present disclosure. In the example of FIG. 4, two users (a first user 410A and a second user 410B) are running together (e.g., relatively near each other over a period of time and distance). The first user 410A is wearing a first wearable device 420A (e.g., wearable device 100 as shown in FIGS. 1 and 2), and the second user 410B is wearing a second wearable device 420B (e.g., another wearable device 100). In other examples, there may be more than two users present, and, in some embodiments, the techniques described herein may be applied to such larger groups as well.

As the users 410A and 410B run, their wearable devices 420A and 420B, respectively, make individualized activity predictions with associated confidence levels. In the example of FIG. 4, the first wearable device 420A may correctly predict that the first user 410A is running with a 90% confidence level (a first activity prediction 430A). The second wearable device may incorrectly predict that the second user 410B is walking with a 60% confidence level (a second activity prediction 430B).

In some embodiments, the wearable devices 420A and 420B may be in communication with each other (e.g., via a wireless communication protocol such as Bluetooth or Wi-Fi). The wearable devices 420A and 420B may communicate to “crowdsource” activity prediction information. For example, the first wearable device 420A may send the first activity prediction 430A or other information to the second wearable device 420B, and the first wearable device 420A may receive the second activity prediction 430B from the second wearable device 420B. Similarly, the second wearable device 420B may receive the first activity prediction 430A from the first wearable device 420A.

After receiving activity prediction 430B, the first wearable device 420A may compare the first activity prediction 430A with the second activity prediction 430B to determine whether to change its prediction. Similarly, after receiving activity prediction 430A, the second wearable device 420B may compare the second activity prediction 430B with the first activity prediction 430A to determine whether to change its prediction.

A wearable device (e.g., the first wearable device 420A) may use any of a variety of techniques for comparing activity predictions. For example, in some embodiments, the device may determine which activity was predicted the most times by members of the group. In the example of FIG. 4, running was predicted once, and walking was predicted once, resulting in a tie between running and walking.

In some embodiments, the device may determine which activity was predicted with the highest average confidence level by members of the group. In the example of FIG. 4, running was predicted with an average confidence level of 90%, and walking was predicted with an average confidence level of 60%, resulting in the first wearable device 420A keeping its prediction set to running, and the second wearable device 420B changing its prediction from walking to running.

In some embodiments, the wearable device may give preference to its own activity prediction by, for example, adjusting or weighting the confidence level of its own activity prediction or adjusting or weighting the confidence levels of received activity predictions. An activity prediction may be biased by, for example, adding or subtracting a predetermined amount to a confidence level (e.g., adding or subtracting 5%, or 10%). A weighting factor may be applied by multiplying a predetermined amount to a confidence level (e.g., multiplying or dividing by 1.05, 1.1, etc.). In the example of FIG. 4, the first wearable device 420A may adjust or weight the first activity prediction 430A from 90% to, e.g., 95% or 100%, and it may adjust or weight the second activity prediction 430B from 60% to, e.g., 55% or 50%.

In the event of a tie, in some embodiments, the device may attempt to break the tie using any of a variety of techniques for breaking the tie. For example, in some embodiments, the wearable device may select from among the aforementioned techniques for comparing activity predictions until one is selected that does not result in a tie. In some embodiments, the wearable device may break a tie by selecting one of the tied activities at random. In other embodiments, the wearable device may break a tie by disregarding one or more of the received (crowdsourced) activity predictions.

These techniques for comparing activity predictions—and for breaking ties that result from certain comparisons—are examples, and a variety of other heuristics, algorithms, or other techniques may be applied to compare or otherwise analyze a set of activity predictions within the group to determine whether to change the activity prediction used by the wearable device during the group activity. In some embodiments, wearable devices in a group may each use different techniques. In some embodiments, the wearable devices may use different firmware or operating system software but implement a common protocol or API to communicate activity predictions to each other. In some embodiments, the wearable device 100 may use a default technique, select an alternative technique, or receive a selection of a technique from a user. In some embodiments, the selected technique may vary depending on the number of users in the group or the number of types of activities detected by devices in the group.

In the example of FIG. 4B, the second user 410B is running, and the second wearable device 420B incorrectly predicted that the second user 410B is walking (with relatively low confidence). Using an average confidence technique, the second wearable device 420B may determine to switch its activity prediction to running. Subsequently, the second wearable device 420B may report running as the detected activity instead of walking to any other components, hardware, firmware, software, apps, etc. in the second wearable device 420B that may make use of that information (e.g., to model calorie expenditure during the activity).

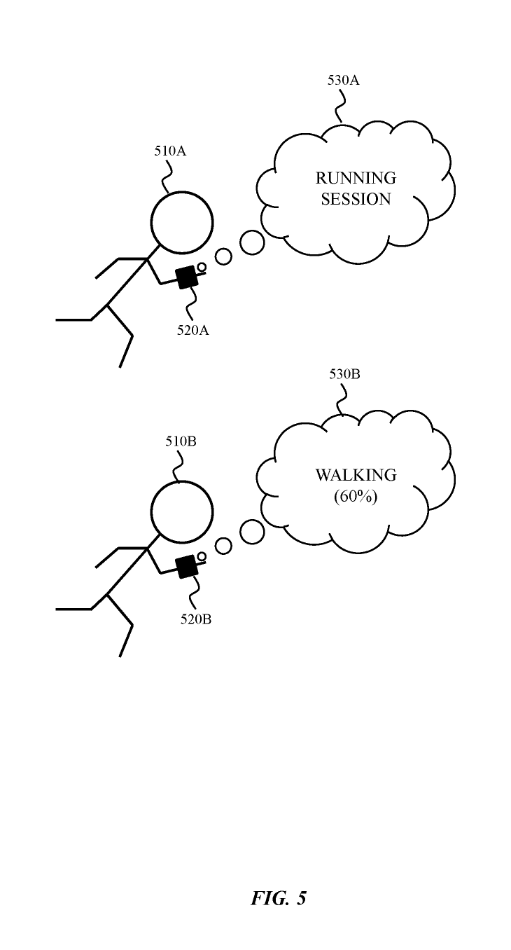

FIG. 5 shows a schematic representation of a group activity in accordance with an embodiment of the present disclosure. In the example of FIG. 5, two users (a first user 510A and a second user 510B) are running together. The first user 510A is wearing a first wearable device 520A, and the second user 510B is wearing a second wearable device 520B.

In some embodiments, a user may indicate to the wearable device 100 that the user is starting (or stopping) an activity session. For example, the user may open an application (“app”) on the wearable device, select an activity (e.g., running or cycling), and tap the display or otherwise indicate to the wearable device 100 that the user is starting the activity. In the example of FIG. 5, the first user 510A has indicated to the first wearable device 520A that the first user 510A is starting a running session (activity session 530A).

The second wearable device 520B may receive information about activity session 530A. In some embodiments, the second wearable device 520B may determine that activity session information from other group members may be given more weight (e.g., the equivalent of a 100% confidence level) or otherwise deemed more reliable than activity predictions within the group.

In the example of FIG. 5, the second wearable device 520B incorrectly predicted the activity of walking with a 60% confidence level (activity prediction 530B). The second wearable device 520B may receive the activity session 530A from the first wearable device 520A and determine that session information from a group member may override prediction information from itself. Consequently, the second wearable device 520B may change its detected activity from walking to running.

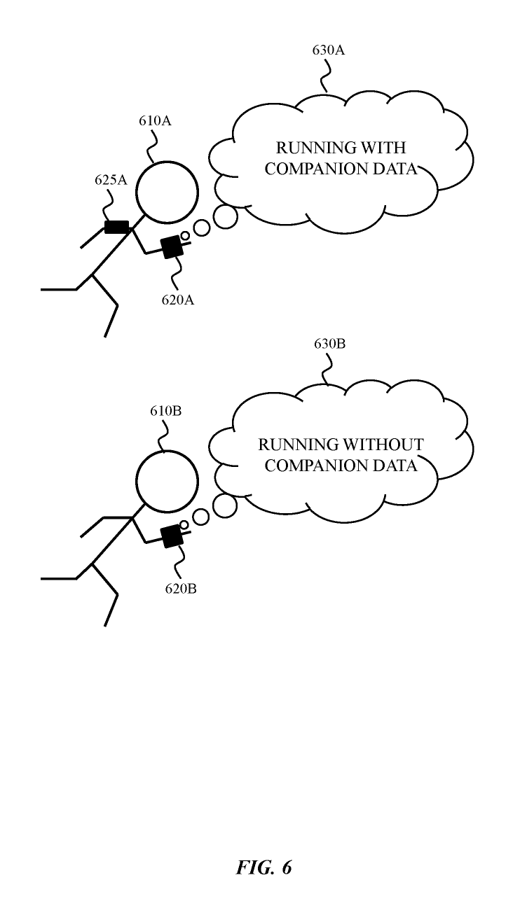

FIG. 6 shows a schematic representation of a group activity in accordance with an embodiment of the present disclosure. In the example of FIG. 6, two users (a first user 610A and a second user 610B) are running together. The first user 610A is wearing a first wearable device 620A, and the second user 610B is wearing a second wearable device 620B. Additionally, the first user 610A is also wearing a companion device 625A, such as the companion device 300 (FIG. 3). In other activities (not shown), a companion device 300 may be mounted on a bicycle or placed in the console of a car or other motor vehicle. As depicted in FIG. 6, the first user 610A is wearing companion device 625A in an armband. The second user 610B is not wearing, carrying, or otherwise in proximity to another companion device 300.

As previously described, the companion device 625A may communicate additional data, including motion data such as GPS data, to the first wearable device 620A. Activity information 630A indicates that the first user 620A is running with companion data, e.g., GPS data.

The second user 610B has no companion device 300 from which the second wearable device 620B could receive companion data. Activity information 630B indicates that the second user 620B is running without companion data.

In some embodiments, wearable devices may communicate additional information such as companion data to other wearable devices in the group. In the example of FIG. 6, the first wearable device 620A may send activity information 630A, including companion data, to the second wearable device 620B. Subsequently, the second wearable device 620B may use the companion data from the companion device 625B. For example, the second wearable device 620B may receive GPS coordinates periodically from the first wearable device 620A and use the GPS coordinates for, e.g., navigation, storing route information, or estimating speed or heading.

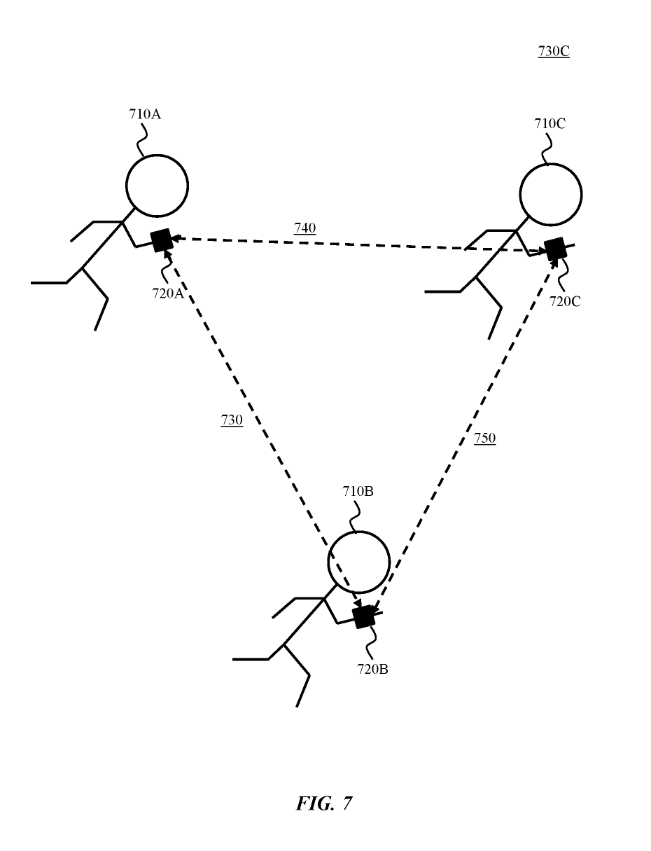

FIG. 7 shows a schematic representation of a group activity in accordance with an embodiment of the present disclosure. In the example of FIG. 7, three users (a first user 710A, a second user 710B, and a third user 710C) are running together. The first user 710A is wearing a first wearable device 720A, the second user 710B is wearing a second wearable device 720B, and a third user 710C is wearing a third wearable device 720C.

As explained above, in some embodiments, groups may include more than two users, and the wearable devices may communicate with each other. In some embodiments, each wearable device 100 may communicate directly with any other wearable device 100 within communicable range. In some embodiments, any of a variety network configurations or topologies may be used (e.g., mesh networks, ring networks, etc.). In some embodiments, each wearable device 100 may route messages to each other via a centralized server or other Internet-connected device.

In the example of FIG. 7, the three wearable devices 720A-C are within range of each other and may communicate directly with each other. These communication channels are depicted as channels 730, 740, and 750. The first wearable device 720A and the second wearable device 720B may communicate with each other over channel 730. The first wearable device 720A and the third wearable device 720C may communicate with each other over channel 740. The second wearable device 720B and the third wearable device 720C may communicate with each other over channel 740.

During an activity session, members of the group may move in and out of range from one another from time to time. For example, if the second user 710B runs faster, and the distance between the first wearable device 720A and the second wearable device 720B exceeds the communicable range between the two devices, the channel 730 may be temporarily unavailable. In some embodiments, each wearable device 100 (e.g., the wearable devices 720A-C) may check periodically to determine which other wearable devices 100 in the group have moved out of range or back into range.

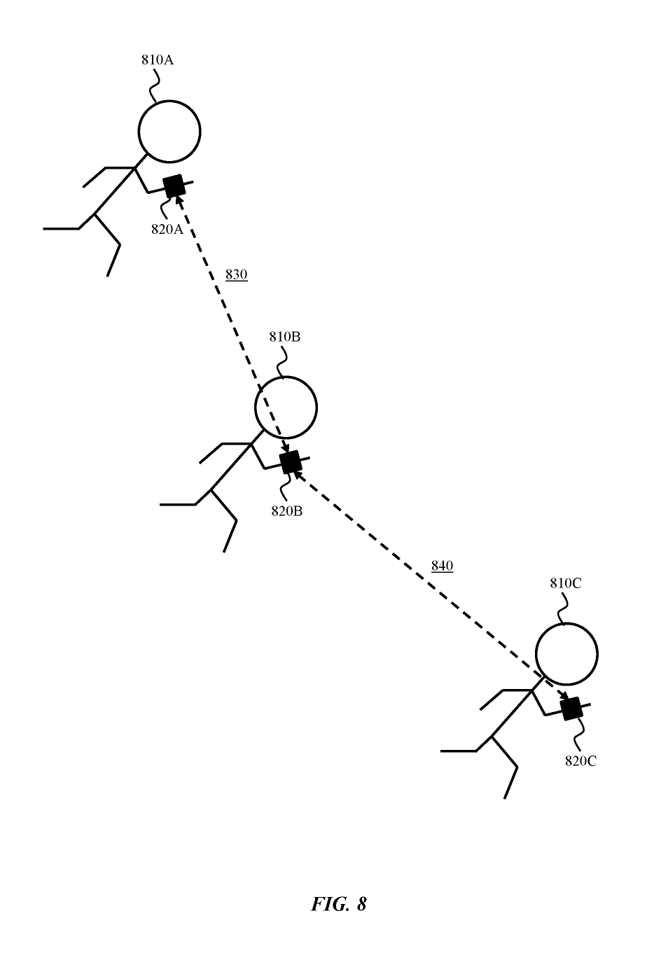

FIG. 8 shows a schematic representation of a group activity in accordance with an embodiment of the present disclosure. In the example of FIG. 8, three users (a first user 810A, a second user 810B, and a third user 810C) are running together. The first user 810A is wearing a first wearable device 820A, the second user 810B is wearing a second wearable device 820B, and a third user 810C is wearing a third wearable device 820C.

As explained above, in some embodiments, each wearable device 100 may communicate directly with any other wearable device 100 within communicable range. In the example of FIG. 8, the first wearable device 820A is in range of the second wearable device 820B and may communicate over channel 830. Similarly, the third wearable device 820C is also in range of the second wearable device 820B and may communicate over channel 840. However, the first wearable device 820A and the second wearable device 820C are not within range of each other and may not communicate directly.

In some embodiments, communications among wearable devices 100 in a group may be limited to one hop. In these embodiments, a first wearable device 100 may only crowdsource activity information from other wearable devices 100 within range of the first wearable device 100. In the example of FIG. 8, the first wearable device 820A may receive activity information from the second wearable device 820B over channel 830 but not the third wearable device 820C. The second wearable device 820B is in range of both the first and third wearable devices 820A and 820C and may receive activity information from either or both via channels 830 and 840, respectively.

In other embodiments, communications among wearable devices 100 in a group may occur over more than one hop. For example, the wearable devices 100 may form an ad hoc network that enables them to route, broadcast, or otherwise retransmit activity information among the wearable devices 100 in the group. In the example of FIG. 8, the first wearable device 820A may receive activity information from the third wearable device 820C (and vice versa) via the second wearable device 820B over both channels 830 and 840.

In some embodiments, a user may join or otherwise select which group or groups to which they belong. For example, the first user 810A may create a group called “My Running Team” and indicate that the second user 810B and the third user 810C should be allowed to join as well. The first user 810A may send an invitation to the second user 810B or the third user 810C, or another user (e.g., 810B) may request to join the group. Users may also leave (or be removed from) groups.

In some embodiments, a user may communicate with any other user who is designated on the first user's contacts or friends list. For example, if the first user 810A is friends or otherwise connected with the second user 810B, their wearable devices 820A and 820B may communicate with each other as part of a group.

In some embodiments, a user may selective enable or disable activity data sharing or other data sharing. For example, if the first user 810A has disabled activity data sharing, the first wearable device 820A may not provide and/or receive crowdsourced activity data with other users, even the other users are within communicable range of the first wearable device 820A.

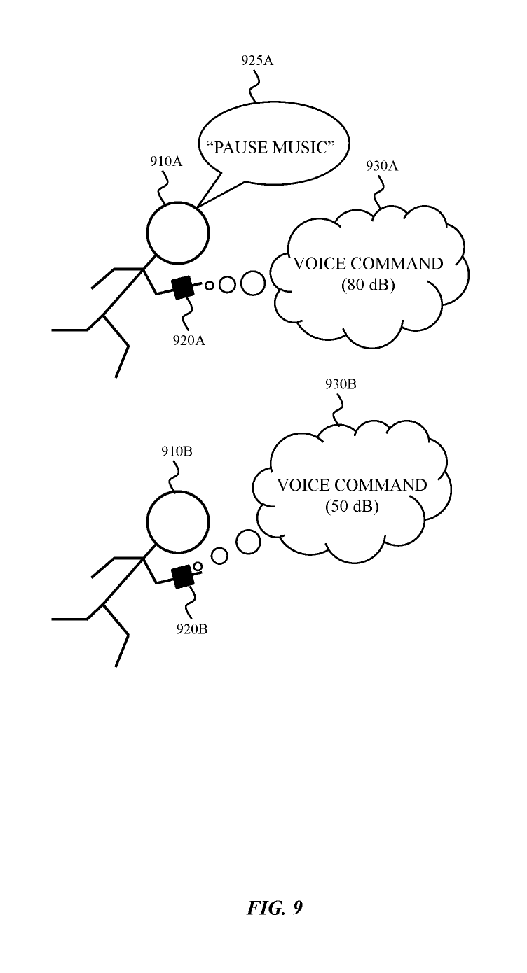

FIG. 9 shows a schematic representation of a group activity in accordance with an embodiment of the present disclosure. In the example of FIG. 9, two users (a first user 910A and a second user 910B) are running together. The first user 910A is wearing a first wearable device 920A, and the second user 910B is wearing a second wearable device 920B.

In some embodiments, communication among wearable devices 100 in a group may transmit other information in addition to, or instead of, activity information (e.g., activity prediction or activity session information) or companion data (e.g., GPS data). This information may be communicated for a variety of purposes to facilitate crowdsourced data sharing.

In the example of FIG. 9, the first user 910A speaks a voice command 925A (e.g., “Pause music”). In some embodiments, any wearable device 100 may hear the voice command 925A but may not necessarily want to respond to the voice command 925A. For example, the second wearable device 920B may hear voice command 925A because it is in proximity to the first user 910A, but the second user 910B may not want the second wearable device 920B to respond to the voice command 925A that was issued by the first user 910A.

In some embodiments, the wearable devices 910A and 920B may determine the power of the voice command. In the example of FIG. 9, the first wearable device 920A determines the power of the voice command to be 80 dB (first voice command power information 930A). The second wearable device 920B, which is farther away from the source of the voice command, determines the power of the voice command to be 50 dB (second voice command power information 930B).

In some embodiments, the first wearable device 920A may receive the second voice command power information 930B from the second wearable device 920B. The first wearable device 920A may compare the relative power (or intensity) of the first and second voice command power information 930A and 930B. The first wearable device 920A may determine that it is closer to the source of the voice command than the second wearable device 920B because the first voice command power information 930A indicated a higher power level than the second voice command power information 930B (80 dB versus 50 dB). The first wearable device 920A may infer that the first user 910A issued the voice command 925A. Consequently, the first wearable device 920A may respond the voice command 925A.

Conversely, the second wearable device 920B may receive the first voice command power information 930A from the first wearable device 920A. The second wearable device 920B may compare the relative power (or intensity) of the first and second voice command power information 930A and 930B. The second wearable device 920B may determine that it is farther from the source of the voice command than the first wearable device 920A because the second voice command power information 930B indicated a lower power level than the first voice command power information 930A (50 dB versus 80 dB). The second wearable device 920B may infer that the first user 910A issued the voice command 925A. Consequently, the second wearable device 920B may ignore the voice command 925A.

Various other information (not shown) may also be crowdsourced or otherwise communicated among wearable devices in a group. For example, the wearable devices may communicate their battery levels among other members of the group. If a first wearable device determines that it has a lower battery level than a second wearable device in the group, it may enter a lower-power mode and rely instead of activity information or other data from the second wearable device with a relatively higher battery level.

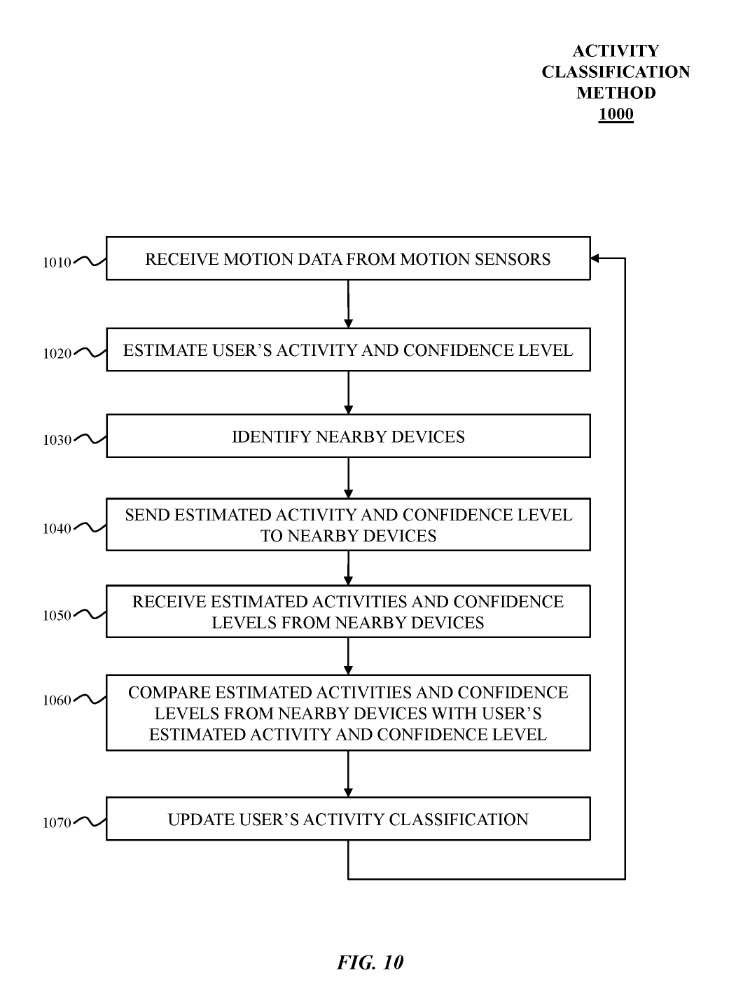

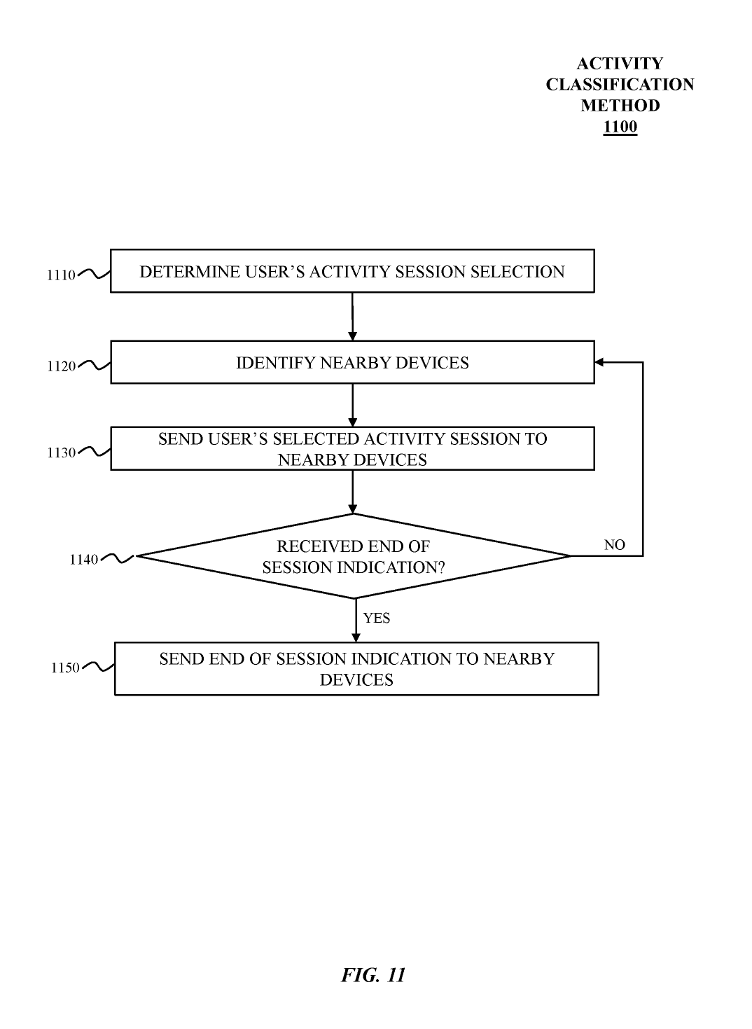

FIG. 10 depicts an activity classification method 1000 in accordance with an embodiment of the present disclosure. Activity classification method 1000 may begin at block 1010.

At block 1010, motion data may be received from motion sensors on a first wearable device (e.g., a wearable device 100) of a first user. In some embodiments, additional motion data or other data (e.g., GPS data) may be received from a companion device (e.g., companion device 300) in communication the first wearable device.

At block 1020, the activity of the first user may be estimated (predicted) by the first wearable device by analyzing the motion data received at block 1010. For example, the first wearable device may determine that the first user is running, walking, cycling, etc. In some embodiments, the confidence level of the activity prediction may also be estimated by the first wearable device. For example, the first wearable device may predict that the first user is cycling with an 85% confidence level.

At block 1030, nearby wearable devices may be identified. In some embodiments, the first wearable device may determine whether any of the nearby devices may be part of the first user's group (e.g., based on a contact list) to facilitate crowdsourcing of activity information or other data.

At block 1040, the predicted activity and estimated confidence level may be sent, by the first wearable device, to the nearby wearable devices.

At block 1050, predicted activities and associated confidence levels may be received from the nearby devices. In some embodiments, the first wearable device may receive information before or after sending information to the nearby devices, or sending and receiving may occur at approximately the same time.

At block 1060, the estimated activities and associated confidence levels received from nearby devices may be compared, by the wearable device, with the user's predicted activity and confidence level. For example, the wearable device may determine the predicted activity with the highest average confidence level.

At block 1070, the user's activity classification (detected activity) may be updated according to the determination made at block 1060. For example, if the wearable device determined that the predicted activity with the highest average confidence level was running at block 1060, the wearable device may change the user's activity classification from the activity predicted at block 1020 to running.

From block 1070, the method may continue at block 1010, repeating the activity classification method 1000 indefinitely until another process or event halts or pauses the method. In other embodiments, the method may end after one iteration, after a predetermined number of iterations, after a predetermined amount of time, etc. In some embodiments, the activity classification method 1000 may pause for a predetermined amount of time before beginning the next iteration at block 1010 so as to, for example, conserve battery power between iterations.

FIG. 11 depicts an activity classification method 1100 in accordance with an embodiment of the present disclosure. Activity classification method 1100 may begin at block 1110.

At block 1110, a first user's activity session selection may be determined by a first wearable device 100. For example, the user may indicate to the first wearable device 100 that the user is beginning a cycling session.

At block 1120, nearby wearable devices may be identified. In some embodiments, the first wearable device may determine whether any of the nearby devices may be part of the first user's group (e.g., based on a contact list) to facilitate crowdsourcing of activity information or other data.

At block 1130, the first user's selected activity session may be sent, by the first wearable device, to the nearby wearable devices. The nearby wearable devices that receive the user's selected activity session may change their activity classifications based on the activity selected by the first user.

At block 1140, the first wearable device 100 may test or otherwise determine whether it has received an end of session indication. For example, the first user may have indicated to the first wearable device that the cycling session has ended.

In some embodiments, if it is determined at block 1140 that the end of session indication has not been received, the activity classification method 1100 may return to block 1120 to, for example, identify nearby wearable devices that may have recently come into range of the group. These recently joined nearby wearable devices may be sent the user's selected activity session.

In other embodiments, the activity classification method 1100 may repeat block 1140 until an end of session indication has been received, or some other process or event halts or pauses the method.

In some embodiments, the activity classification method 1100 may pause for a predetermined amount of time before beginning the next iteration at block 1120 (or block 1140, etc.) so as to, for example, conserve battery power between iterations.

In some embodiments, if it is determined at block 1140 that the end of session indication has been received, the activity classification method 1100 may proceed to block 1150.

At block 1150, the end of session indication may be sent, by the first wearable device, to the nearby wearable devices. The nearby wearable devices that receive the end of session indication may change their activity classifications based on, for example, an activity predicted by the device, or based on a comparison of crowdsourced activity information as in activity classification method 1000.

In some embodiments, after block 1150, activity classification method 1100 may end.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Further, although the present disclosure has been described herein in the context of at least one particular implementation in at least one particular environment for at least one particular purpose, those of ordinary skill in the art will recognize that its usefulness is not limited thereto and that the present disclosure may be beneficially implemented in any number of environments for any number of purposes.

1. A method comprising:
predicting, by a first wearable device, a first predicted activity of the first user using motion data received by motion sensors of the first wearable device;
estimating, by the first wearable device, a first confidence level of the first predicted activity;
receiving, by the first wearable device over a wireless communication channel from a second wearable device, a second predicted activity of a second user and a second confidence level of the second predicted activity;
comparing, by the first wearable device, the first predicted activity and the first confidence level with the second predicted activity and the second confidence level; and
determining, by the first wearable device, a first activity classification for the first user to be the second predicted activity when a second average confidence level associated with the second predicted activity is greater than a first average confidence level associated with the first predicted activity.

2. The method of claim 1, wherein predicting is triggered by a first user action on a display of the first wearable device.

3. The method of claim 1, wherein predicting is triggered by receiving a voice command of the first user.

4. The method of claim 1, wherein motion data comprises accelerometer data.

5. The method of claim 1, wherein the second predicted activity is received from the second wireless device via one or more relaying devices.

6. The method of claim 5, wherein at least one of the relaying devices is a companion device.

7. The method of claim 1, wherein the predicting further uses positioning data received from a companion device.

8. The method of claim 7, wherein the positioning data is GPS data.

9. The method of claim 1, wherein the first confidence level is adjusted prior to comparing the first confidence level and second confidence level.

10. The method of claim 1, further comprising:
receiving, by the first wearable device over a wireless communication channel from a plurality of additional wearable devices, a plurality of predicted activities of a plurality of additional users and a corresponding plurality of confidence levels of the plurality of predicted activities;
wherein comparing further comprises comparing, by the first wearable device, the first predicted activity and the first confidence level with at least some of the plurality of predicted activities and the corresponding confidence levels; and
wherein determining the first activity classification is further based on the comparison of the first predicted activity and the first confidence level with at least some of the plurality of predicted activities and the corresponding confidence levels.

11. An apparatus comprising:
a display;
one or more motion sensors;
a wireless interface; and
at least one processor, wherein the at least one processor is configured to:
receive motion data from the one or more motion sensors;
predict a first predicted activity of the user of the apparatus using the received motion data;
estimate a first confidence level of the first predicted activity;
receive, via the wireless interface, a second predicted activity of a user of a second apparatus and a second confidence level of the second predicted activity;
compare the first predicted activity and the first confidence level with the second predicted activity and the second confidence level; and
determine a first activity classification for the first user to be the second predicted activity when a second average confidence level associated with the second predicted activity is greater than a first average confidence level associated with the first predicted activity.

12. The apparatus of claim 11, wherein the apparatus is a wrist-worn device.

13. The apparatus of claim 11, wherein the wireless interface is a mesh wireless network interface.

14. The apparatus of claim 11, wherein the wireless interface is a IEEE 802.15 interface.

15. The apparatus of claim 11, wherein the wireless interface connects to a companion device and wherein the companion device is a smartphone.

16. The apparatus of claim 11, further comprising a heart rate sensor, wherein the first predicted activity of the user is further predicted based on heart rate data of the user of the apparatus received by the at least one processor from the heart rate sensor.

17. The apparatus of claim 11, further comprising a microphone, wherein the at least one processor is further configured to trigger prediction after receiving a voice command of the user of the apparatus received by the at least one processor from the microphone.

18. The apparatus of claim 11, further comprising one or more of a barometer and altimeter, wherein the first predicted activity of the user is further predicted based on altitude data received by the at least one processor from the one or more of a barometer and altimeter.

19. The apparatus of claim 11, wherein at least one of the one or more motion sensors is a gyroscope.

20. The apparatus of claim 11, further comprising a GPS receiver, wherein the first predicted activity of the user is further predicted based on positioning data received by the at least one processor from the GPS receiver.
